(19) United States
(12) Patent Application Publication
(10) Pub. No.: US 2025/0325880 A1
(43) Pub. Date: Oct. 23, 2025

(54) TOUCH SENSING SPORT BALL SYSTEM AND RELATED METHODS OF USE

(71) Applicant: NIKE, Inc., Beaverton, OR (US)

(72) Inventor: Arthur P. MOLINARI, Portland, OR (US)

(73) Assignee: NIKE, Inc., Beaverton, OR (US)

(21) Appl. No.: 19/045,167

(22) Filed: Feb. 4, 2025

Related U.S. Application Data
(60) Provisional application No. 63/635,948, filed on Apr. 18, 2024.

(51) Int. Cl. A63B 43/00; A63B 41/02; A63B 41/08; A63B 71/06

(52) U.S. Cl. CPC A63B 43/00; A63B 41/02; A63B 41/08; A63B 71/0619; A63B 2220/53; A63B 2220/56; A63B 2220/58; A63B 2220/833; A63B 2225/20; A63B 2225/62; A63B 2243/0025

(57) ABSTRACT

In one instance, disclosed herein is a touch sensing sport ball system, including a casing that forms an exterior layer of a sport ball; a bladder disposed within the casing; a touch sensing layer disposed between the exterior layer and the bladder and operative to generate touch data when the sport ball is impacted; and at least one processor operative to: access the touch data generated by the touch sensing layer; determine, based at least in part on the touch data, that the sport ball has been impacted; and output an indication that the sport ball has been impacted.

### CROSS-REFERENCE TO RELATED APPLICATION(S)

This patent application claims the benefit of priority to U.S. Provisional Application No. 63/635,948, filed on Apr. 18, 2024, the entirety of which is incorporated herein by reference.

### TECHNICAL FIELD

The present disclosure relates generally to sport balls, and, more particularly, to a sport ball capable of analyzing impacts of the sport ball.

### BACKGROUND

A variety of inflatable sport balls (e.g., soccer balls, footballs, basketballs, etc.) typically include a casing and a hollow bladder. The casing of a sport ball is generally formed from a durable, wear-resistant material. An exterior surface of the casing forms an exterior layer of the sport ball. The bladder is generally formed from a stretchable material. The bladder of the sport ball is disposed within the casing and configured to be filled or inflated with a fluid, such as air. When the bladder is inflated, the pressurized bladder exerts an outward force against an interior surface of the casing, thereby giving the sport ball a persistent shape when at rest. The shape of the sport ball may be deformed, however, such as when the sport ball is compressed or otherwise impacted by another object.

### SUMMARY

In one aspect, a touch sensing sport ball system includes: a casing that forms an exterior layer of a sport ball; a bladder disposed within the casing; a touch sensing layer disposed between the exterior layer and the bladder and operative to generate touch data when the sport ball is impacted; and at least one processor operative to: access the touch data generated by the touch sensing layer; determine, based at least in part on the touch data, that the sport ball has been impacted; and output an indication that the sport ball has been impacted.

In another aspect, a method for analyzing an impact of a sport ball includes: generating touch data in response to a sport ball being impacted; determining, based at least in part on the touch data generated in response to the sport ball being impacted, that the sport ball has been impacted; and causing a graphical user interface (GUI) to display a visual indication that the sport ball has been impacted.

In another aspect, a touch sensing sport ball apparatus includes: a casing that forms an exterior layer of a sport ball; a bladder disposed within the casing; and a touch sensing layer disposed between the exterior layer and the bladder and operative to generate touch data when the sport ball is impacted that can be used by at least one processor to determine that the sport ball has been impacted.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings, of which:

FIG. 1 illustrates a perspective view of a sport ball including a touch sensing layer;

FIG. 2 depicts a diagram of a touch sensing sport ball system;

FIG. 3A illustrates an impact of a sport ball against a surface;

FIG. 3B depicts touch data generated by a touch sensing layer in response to an impact of a sport ball against a surface;

FIG. 4A illustrates an impact of a sport ball by an animated member;

FIG. 4B depicts touch data generated by a touch sensing layer in response to an impact of a sport ball by an animated member;

FIG. 5A illustrates an impact of a sport ball against a surface;

FIG. 5B depicts touch data generated by a touch sensing layer in response to an impact of a sport ball against a surface;

FIG. 6A illustrates an impact of a sport ball by an animated member;

FIG. 6B depicts touch data generated by a touch sensing layer in response to an impact of a sport ball by an animated member;

FIGS. 7A and 7B depict touch hystereses representing impacts of a sport ball against one or more surfaces and by one or more animated members

FIG. 8 illustrates a graphical user interface displaying touch data; and

FIG. 9 depicts a flow diagram of a method for determining that a sport ball has been impacted by an animated member.

### DETAILED DESCRIPTION

Both the foregoing general description and the following detailed description are exemplary and explanatory only and are not restrictive of the features as claimed. As used herein, the terms “comprises,” “comprising,” “having,” including,” or other variations thereof, are intended to cover a non-exclusive inclusion such that a process, method, system, article, or apparatus that comprises a list of elements does not necessarily include only those elements, but may include other elements not expressly listed or inherent to such a process, method, system, article, or apparatus. Further, relative terms, such as, for example, “about,” “substantially,” “generally,” and “approximately” are used to indicate a possible variation of ±10% in a stated value. While various features and functions of the present disclosure are described herein in the context of soccer balls, it will be understood that various features and functions of the present disclosure may be applied in the context of many different types of sport balls.

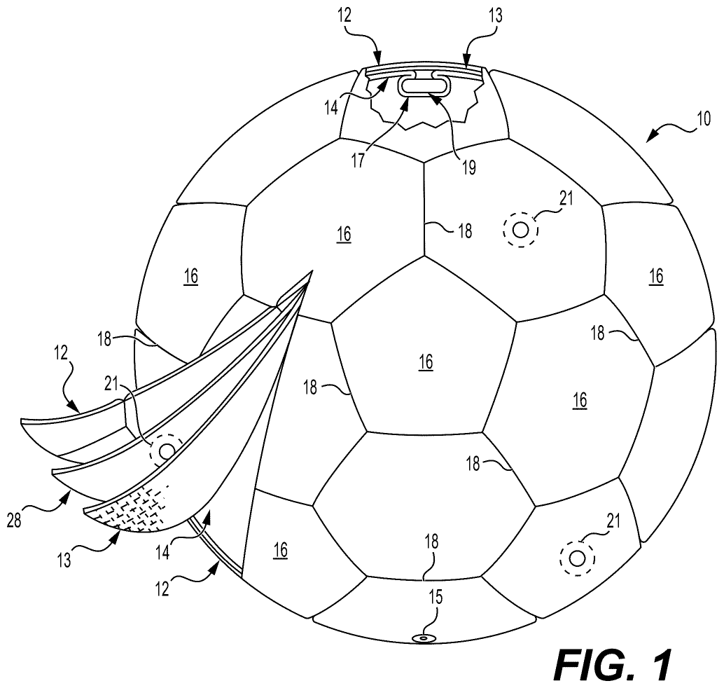

FIG. 1 illustrates a first perspective view of a sport ball including a touch sensing layer. As illustrated in FIG. 1, a sport ball 10 has a layered structure that includes at least an exterior casing 12 and an interior bladder 14. As mentioned above, a casing 12 forms an exterior (e.g., an exterior layer) of the sport ball 10. In some instances, as illustrated in FIG. 1, a casing 12 includes two or more panels 16 that are stitched, adhered, bonded, welded, or otherwise joined together along abutting sides or edges, forming one or more seams 18. Often, as illustrated in FIG. 1, panels 16 are pentagonal or hexagonal in shape. However, in other instances, panels 16 may have non-equilateral shapes, non-regular or non-geometric shapes, or a variety of other shapes that combine in a tessellation-type manner to form the casing 12.

In some instances, the panels 16 of a casing 12 are all of the same shape (e.g., hexagonal). In some instances, the panels 16 of a casing 12 include two or more different shapes (e.g., hexagons and pentagons). The abutting sides of the panels 16 that combine to form the seams 18 may be linear, concave, convex, or otherwise non-linear edges. In some instances, a casing 12 may have a seamless structure, such that the casing 12 has no distinct panels 16 and no seams 18. In some such instances, a casing 12 may be formed by a single piece of material. Accordingly, the construction of a casing 12 may vary significantly, leading to a wide variety of configurations of panels 16. For example, many modern soccer balls include twelve pentagonal panels 16 and twenty hexagonal panels 16. Or for example, the four panels 16 of modern American footballs are pointed ellipses (sometimes referred to as a “marquise” shape).

The material(s) selected for a casing 12, or for an individual panel 16, may be leather, synthetic leather, polyurethane, polyvinyl chloride, rubber, or any other suitable material that is generally durable and wear-resistant. In some instances, each panel 16 of a sport ball 10 may include two or more layers different materials. For example, in some instances, each panel 16 included in a casing 12 may include a polymer foam layer and a non-foamed polymer layer. Or for example, in some instances, a panel 16 of a casing 12 may include an exterior polyvinyl chloride layer, an interior textile layer, and an intervening polymer foam layer.

As mentioned above, a bladder 14 of a sport ball 10 is typically hollow and disposed within a casing 12. The bladder 14 is typically formed from a stretchable material and configured to be filled or inflated with a fluid, such as air. For example, in some instances, the bladder 14 may be formed from a rubber or carbon latex material that substantially prevents air or other fluids contained within the bladder 14 from diffusing through the material. However, the bladder 14 may be formed using a variety of other polymer or elastomeric materials.

In order to facilitate inflation, the bladder 14 typically includes a valve 15 that extends from the bladder 14 and through the casing 12, thereby being accessible from outside of the sport ball 10. However, in some instances, a bladder 14 may have a valve-less structure that is semi-permanently inflated. When inflated, the bladder 14 becomes pressurized and exerts an outward force against an interior surface of the casing 12, thereby giving the sport ball 10 a persistent shape, generally determined by the shape or configuration of the casing 12, when the sport ball 10 is at rest. However, the shape of the sport ball 10 when the sport ball 10 is at rest may be determined at least in part by the shape or configuration of the casing 12, the shape or configuration of the bladder 14, or the shape or configuration of an intervening restriction layer 13, as described below. For example, as illustrated in FIG. 1, the configuration of the pentagonal and hexagonal panels 16 of the casing 12 give the sport ball 10 a spherical shape when the bladder 14 is inflated. Or for example, the pointed ellipse (e.g., marquise) shape of the panels of an American football give an American football its ovoid shape when the bladder 14 of the American football is inflated.

In some instances, as illustrated in FIG. 1, the bladder 14 includes a pocket 17. A pocket 17 included in a bladder 14 may provide a cavity, indentation, void, or other space that receives and holds a component 19, such as a device or a counterweight. In some instances, as illustrated in FIG. 1, when a bladder 14 is disposed within a casing 12 of a sport ball 10, the pocket 17 included in the bladder 14 protrudes or projects inward and toward a center of the sport ball 10, thereby locating a component 19 included in the pocket 17 within an interior area of the sport ball 10. In this position, the component 19 is protected from impacts of the sport ball 10 with surfaces, animated members, or other objects when the sport ball 10 is being utilized. The shape and size of a pocket 17 may be selected to accommodate a component 19, such that the pocket 17 receives and securely retains the component 19 within the sport ball 10.

A component 19 may include one or more electronic devices, such as a microprocessor, transmitter, receiver, memory, battery, or any other combination of elements that process, send, receive, or collect data. More specifically, examples of electronic devices that might be included in a component 19 include one or more of a) a pressure sensor for determining the pressure of a fluid contained within the bladder 14; b) a global positioning system (GPS) unit and/or an accelerometer that measures various factors relating to the location or movement the sport ball 10; c) a line sensor that determines whether the sport ball 10 has crossed a goal line or an out-of-bounds line; d) a radio-frequency identification (RFID) chip that stores data relating to the sport ball 10 or assists with identifying the sport ball 10; and e) a camera that collects image data. A component 19 may additionally or alternatively include a counterweight in order to enhance the balance, weight distribution, center of mass, or other properties of a sport ball 10. In many instances, one or more electronic devices included in a component 19 may also serve as a counterweight. However, it is contemplated that in other embodiments no pocket 17 or component 19 is present in bladder 14.

In some instances, as illustrated in FIG. 1, a sport ball 10 also includes a restriction layer 13. As illustrated in FIG. 1, a restriction layer 13 forms a middle layer of a sport ball 10 and is positioned between a casing 12 and a bladder 14. In general, a restriction layer 13 is formed from materials with a limited degree of stretch in order to restrict expansion of the bladder 14. For example, a restriction layer 13 may be formed from a) a thread, yarn, or filament that is repeatedly wound around a bladder 14 in various directions to form a mesh that covers substantially all of the bladder 14; b) a plurality of generally flat or planar textile elements stitched together to form a structure that extends around a bladder 14; c) a plurality of generally flat or planar textile strips that are impregnated with latex and placed in an overlapping configuration around a bladder 14; or d) a substantially seamless spherically-shaped textile. In some instances, a restriction layer 13 may also be bonded, joined, or otherwise incorporated into either of a casing 12 or a bladder 14. However, in some instances, a sport ball 10 need not include a restriction layer 13.

In some instances, as illustrated in FIG. 1, a sport ball 10 includes one or more touch sensors 21. A touch sensor 21 may be any sensor capable of detecting when the sport ball 10 is impacted. For example, in some instances, a touch sensor 21 may be an electromechanical sensor (e.g., a piezoelectric sensor or device) configured to generate a voltage in response to a physical force. In such an instance, when the sport ball 10 is impacted, an electromechanical touch sensor 21 included in the sport ball 10 may generate a voltage in response to the impact. The voltage generated by the electromechanical touch sensor 21 may be proportional to the magnitude of the physical force. Or for example, in some instances, a touch sensor 21 may be an electromechanical sensor (e.g., a strain gauge) configured to measure a change in the resistance of a conductive material through which a standing current is passed in response to a physical force. The standing current may be supplied by a battery housed in the pocket 17 included in the bladder 14 of the sport ball 10. In such an instance, when the sport ball 10 is impacted, an electromechanical touch sensor 21 included in the sport ball 10 may generate or record a change in resistance in response to the impact. The change in resistance generated or recorded by the electromechanical touch sensor 21 may be proportional to the magnitude of the physical force. As described below, a voltage or a change in resistance generated or recorded by one or more touch sensors 21 included in the sport ball 10 may be used by one or more processors 22 to determine, for example, if and when the sport ball 10 impacted, how hard the sport ball 10 was impacted, a shape of an object that impacted by the sport ball 10, or whether the sport ball 10 was impacted by a surface 30 or an animated member 60. However, a touch sensor 21 may be any other suitable sensor. Information generated by a touch sensor 21 (e.g., voltages or changes in resistance) may be referred to as “touch data.” The sport ball 10 may include any internal circuitry necessary to communicatively couple a touch sensor 21 with any other component of the sport ball 10, such as, for example, a battery operative to supply electricity to a touch sensor 21, a memory operative to receive and store touch data generated by a touch sensor 21, a communication component operative to transmit touch data generated by a touch sensor 21 to one or more processors 22, or one or more processors 22 housed within a pocket 17 of the bladder 14 of the sport ball 10 and operative to receive and process touch data generated by a touch sensor 21.

As mentioned above, a sport ball 10 may include one or more touch sensors 21. For example, in some instances, as illustrated in FIG. 1, one or more touch sensors 21 are integrated into or otherwise disposed within the casing 12 of the sport ball 10. Or for example, in some instances, as illustrated in FIG. 1, a sport ball 10 includes a touch sensing layer 28 that includes one or more touch sensors 21. In some instances, a touch sensing layer 28 is disposed within the sport ball 10 immediately beneath the casing 12 of the sport ball 10. In such an instance, the touch sensing layer 28 may cover only a part of the interior surface of the sport ball 10, or the touch sensing layer 28 may cover the entirety of the interior surface of the casing 12 of the sport ball 10. In some instances, a touch sensing layer 28 is coupled to or otherwise in contact with a restriction layer 13 of the sport ball 10 (e.g., when the sport ball 10 is inflated). A touch sensing layer 28 may be disposed within the sport ball 10 inward or outward of a restriction layer 13. In some instances, when one or more touch sensors 21 are integrated into or otherwise disposed within the casing 12 of the sport ball 10, the casing 12 itself forms a touch sensing layer 28. Similarly, in some instances, one or more touch sensors 21 may be integrated into or otherwise disposed within a restriction layer 13 of the sport ball 10, such that the restriction layer 13 itself forms a touch sensing layer 28.

In some instances, a touch sensing layer 28 may be formed entirely or in part from a conductive material or a piezoelectric material, such that the touch sensing layer 28 itself may function as an electromechanical touch sensor 21, as described above. In such an instance, no matter where or how a sport ball 10 that includes the touch sensing layer 28 is impacted, the touch sensing layer 28 is able to accurately measure a force of the impact, unlike an accelerometer, for which the measure of a force of an impact of a sport ball may be influenced by where the sport ball was impacted relative to a position of the accelerometer. Piezoelectric materials are a group of materials that generate an electric potential difference upon application of a mechanical force. In response to an applied force, a voltage is generated in the piezoelectric material that is proportional to the applied force. One common piezoelectric material is quartz, which is typically used in watches. Many other natural and synthetic materials are piezoelectric, including various crystals, ceramics, and polymers. A piezoelectric polymer may include, but is not limited to: polyvinyl fluoride (PVF), polyvinylidene fluoride (PVDF), polyvinyl chloride (PVC), polytetra-fluoroethylene-polyvinylidene fluoride (PTFE-PVF2) and other polymers, copolymers, and ceramic polymer mixtures.

In some instances, a touch sensing layer 28 includes a plurality of touch sensors 21 that correspond to a respective plurality of locations along the exterior surface of a casing 12 of a sport ball 10 and combine to form a lattice 29 of touch sensors 21. In such an instance, because each touch sensor 21 included in the lattice 29 of touch sensors 21 corresponds with a unique location along the exterior surface of the casing 12, touch data 26 generated by the touch sensors 21 can be used in conjunction with the locations of the touch sensors 21 to determine a shape of an object that impacted the sport ball 10, or whether the sport ball 10 was impacted by a surface 30 or an animated member 60, as described below.

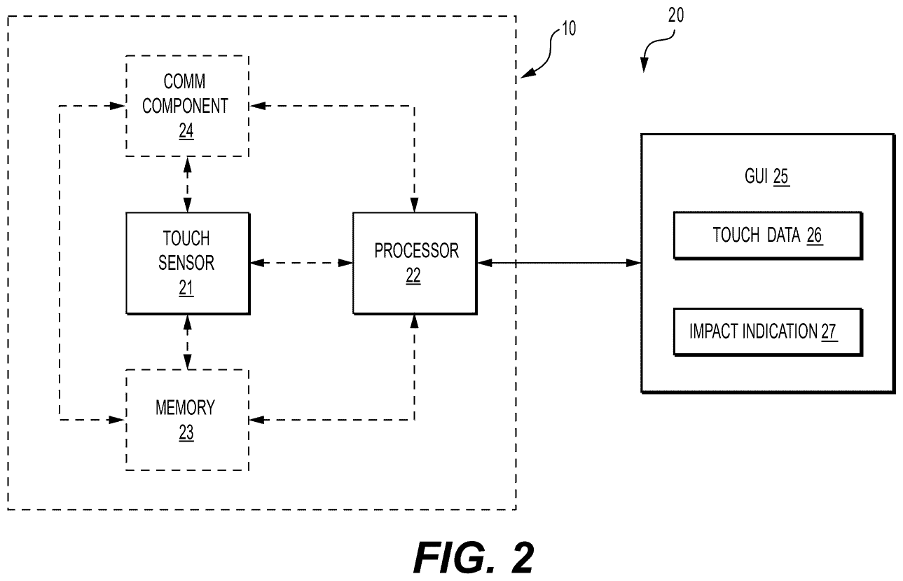

FIG. 2 depicts a diagram of a touch sensing sport ball system. In some instances, as depicted in FIG. 2, a touch sensing sport ball system 20 includes a sport ball 10, one or more touch sensors 21 disposed within the sport ball 10, and a processor 22. In general, the sport ball 10, the one or more touch sensors 21, and the processor 22 of the touch sensing sport ball system 20 function cooperatively to determine a) if and when the sport ball 10 is impacted; b) how hard the sport ball 10 is impacted; and/or c) what the sport ball 10 is impacted by, e.g., a surface 30 or an animated member 60. In some instances, as depicted in FIG. 2, the touch sensing sport ball system 20 additionally includes a computer-readable memory 23, a communication component 24, or a graphical user interface (GUI) 25. In some instances, in addition to the one or more touch sensors 21, one or more of the processor 22, the computer-readable memory 23, and the communication component 24 may also be disposed within the sport ball 10.

As mentioned above, one or more touch sensors 21 may be disposed within the sport ball 10. For example, a touch sensor 21 may be disposed within a casing 12 of the sport ball 10, or within a touch sensing layer 28, as described above. However a touch sensor 21 is disposed within the sport ball 10, a touch sensor 21 is operative to generate computer-readable touch data 26 in response to an impact of the sport ball 10. For example, in some instances, as described above, a touch sensor 21 is an electromechanical sensor operative to generate touch data 26 by generating a voltage or recording a change in resistance in response to an impact of the sport ball 10. In some instances, a touch sensor 21 is operative to generate touch data 26 only when the touch data 26 would be significant, e.g., if a voltage generated by a touch sensor 21 would exceed a voltage threshold, or if a change in resistance recorded by a touch sensor 21 would exceed a resistance threshold. However, a touch sensor 21 may be operative or configured to generate touch data 26 in response to an impact of a sport ball 10 in any other way. In some instances, as depicted in FIG. 2, a touch sensor 21, or a touch sensing layer 28 including one or more touch sensors 21, is communicatively coupled to the processor 22, the memory 23, or the communication component 24. In some such instances, after generating touch data 26, a touch sensor 21 is operative to transmit or otherwise provide the touch data 26 to the processor 22, the memory 23, or the communication component 24.

The processor 22 is a computing device that is operative to receive touch data 26 generated by one or more touch sensors 21 and determine, using or based at least in part on the touch data 26, if the sport ball 10 has been impacted, how hard the sport ball has been impacted, or what the sport ball 10 has been impacted by, as described in further detail below. In some instances, the processor 22 is further operative to cause a GUI 25 to display touch data 26 generated by one or more touch sensors 21 or an impact indication 27, e.g., an indication that the sport ball 10 has been impacted, which may further include an indication of how hard the sport ball 10 was impacted or an indication of what the sport ball 10 was impacted by. In some instances, as depicted in FIG. 2, the processor 22 is disposed within the sport ball 10 and communicatively coupled to one or more touch sensors 21, such that the processor 22 can receive touch data 26 generated by the one or more touch sensors 21 directly. In some instances, however, the processor 22 receives touch data 26 generated by one or more touch sensors 21 indirectly. For example, in some instances, a computer-readable memory 23 is communicatively coupled to both the one or more touch sensors 21 and the processor 22. In some such instances, the one or more touch sensors 21 are operative to transmit or otherwise provide touch data 26 to the computer-readable memory 23, and the processor 22 is operative to access the touch data 26 from the computer-readable memory 23. Or for example, in some instances, the processor 22 is not disposed within the sport ball 10. In some such instances, a communication component 24 is communicatively coupled to the one or more touch sensors 21 or a computer-readable memory 23 that is communicatively coupled to the one or more touch sensors 21, and the communication component 24 is operative to receive touch data 26 from the one or more touch sensors 21 or the computer-readable memory 23 and provide the touch data 26 to the processor 22 by establishing a physical or wireless communication link with the processor 22.

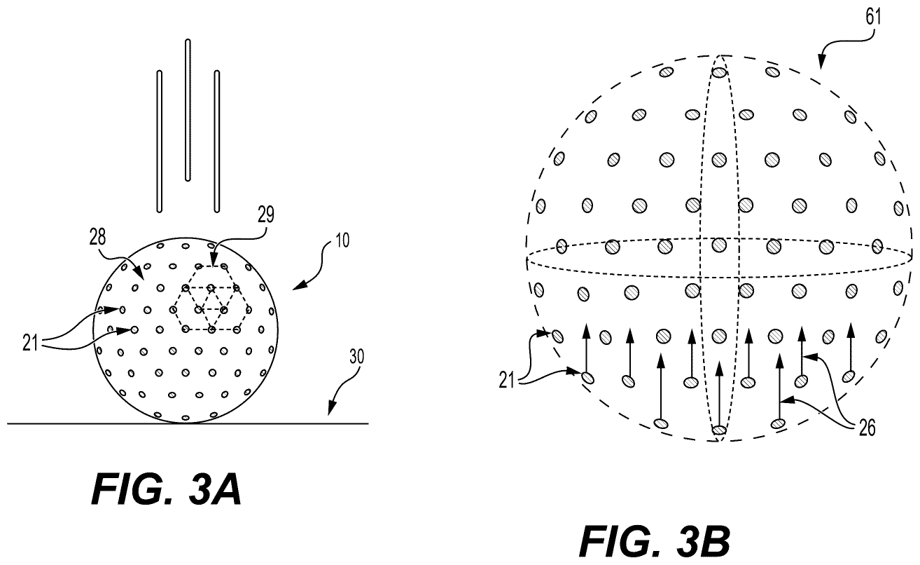

As mentioned above, in various instances, a touch sensing sport ball system 20 is operative to determine a) if and when a sport ball 10 is impacted; b) how hard a sport ball 10 is impacted; and/or c) what a sport ball 10 is impacted by, e.g., a surface or an animated member. FIG. 3A illustrates an impact of a sport ball 10 against a surface 30. A surface 30 may be a substantially flat surface or any other stationary or unanimated object, whether that object is substantially flat or not. For example, a surface 30 may be the pitch of a soccer field or a goalpost of a soccer goal. In the example illustrated in FIG. 3A, the sport ball 10 is a soccer ball that includes a casing 12, a bladder 14 disposed within the casing 12, and a touch sensing layer 28 including a plurality of touch sensors 21 and disposed between the casing 12 and the bladder 14, as described above. The bladder 14 of the sport ball 10 is inflated with air. Pressure exerted by the inflated bladder 14 against the interior surface of the casing 12 provides the sport ball 10 with a persistent shape while at rest. When the sport ball 10 is impacted, however, the shape of the sport ball 10 may be deformed, however momentarily, allowing the object that impacted the sport ball 10 to contact the sport ball 10 at multiple (e.g., adjacent) locations along the exterior surface of the casing 12 of the sport ball 10. In this example, the plurality of touch sensors 21 included in the touch sensing layer 28 combine to form a lattice 29 of touch sensors 21. Although the lattice 29 is illustrated in FIG. 3A as including only some of the touch sensors 21 included in the touch sensing layer 28, for simplicity, it should be understood that the lattice 29 may include all of the touch sensors 21 included in the touch sensing layer 28.

FIG. 3B depicts an example of touch data 26 generated by a plurality of touch sensors 21 in response to an impact of a sport ball 10 being impacted against a surface 30, as illustrated in FIG. 3A. In the example depicted in FIG. 3B, touch sensors 21 with arrows projecting away from them represent touch sensors 21 corresponding to locations along the exterior surface of the casing 12 of the sport ball 10 at which the casing 12 of the sport ball 10 was impacted by the surface 30. In this example, the length of the arrow projecting away from a touch sensor 21 represents the magnitude of the touch data 26 generated by the touch sensor 21 (e.g., the magnitude of the voltage or change in resistance generated by an electromechanical touch sensor 21, as described above). For example, in the example depicted in FIG. 3B, each touch sensor 21 of the bottom two circles of touch sensors 21 (depicted in FIG. 3B as rows, in two dimensions) of the lattice 29 of touch sensors 21 corresponds to a location along the exterior surface of the casing 12 of the sport ball 10 at which the casing 12 was impacted by the flat surface 30, as illustrated in FIG. 3A. The three-dimensional map of touch sensors 21 corresponding to locations along the exterior surface of the casing 12 of the sport ball 10 at which the sport ball 10 was impacted (e.g., the touch sensors 21 represented by black circles; in this example, the bottom two rows of touch sensors 21) may be referred to as an “impact map” 61. As depicted in FIG. 3B, an impact map 61 may include a map (e.g., a three-dimensional map) of touch sensors 21 corresponding to locations along the exterior surface of a casing 12 of a sport ball 10 at which the sport ball 10 was impacted, and touch data 26 generated by those touch sensors 21.

As depicted in FIG. 3B, the magnitude of the touch data 26 generated by the touch sensors 21 of the bottom circle of touch sensors 21 is greater than the magnitude of the touch data 26 generated by the touch sensors 21 of the second to bottom row of touch sensors 21. This is because the shape of the sport ball 10 would have had to be deformed (e.g., flattened) by the force of the flat surface 30 impacting the sport ball 10 at the locations along the exterior surface of the casing 12 of the sport ball 10 corresponding to the bottom row of touch sensors 21 before the flat surface 30 would have been able to impact the sport ball 10 at the locations along the exterior surface of the casing 12 corresponding to the second to bottom row of touch sensors 21, thereby dampening the force applied by the impact of the sport ball 10 with the flat surface 30 at the locations along the exterior surface of the casing 12 corresponding to the second to bottom row of touch sensors 21, relative to the force applied by the impact of the sport ball 10 with the flat surface 30 at the locations along the exterior surface of the casing 12 corresponding to the bottom row of sensors 21.

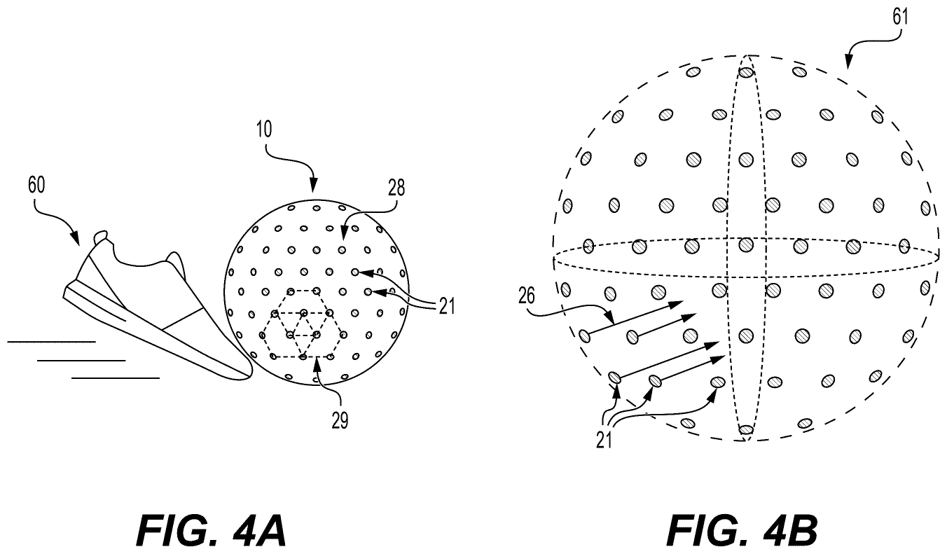

Similarly, FIG. 4A illustrates an impact of a sport ball 10 by an animated member 60, and FIG. 4B depicts an example of an impact map 61 including touch data 26 generated by a plurality of touch sensors 21 included in the sport ball 10 in response to the impact of the sport ball 10 by the animated member 60. In the examples illustrated and depicted by FIGS. 4A and 4B, because the shape of the animated member 60 is small and round, compared to the shape of the flat surface 30, the sport ball 10 is impacted at fewer locations along the exterior casing 12 of the sport ball 10, corresponding to fewer touch sensors 21, accordingly, and producing an impact map 61 with a different shape. Similar to the example depicted in FIG. 3B, the shape of the sport ball 10 would have had to be deformed by the force of the animated member 60 impacting the sport ball 10 at the locations along the exterior surface of the casing 12 of the sport ball 10 corresponding the left two touch sensors 21 before the animated member 60 would have been able to impact the sport ball 10 at the locations along the exterior surface of the casing 12 corresponding to the right two touch sensors 21, thereby dampening the force applied by the impact of the sport ball 10 by the animated member 60 at the locations along the exterior surface of the casing 12 corresponding to the right two touch sensors, relative to the force applied by the impact of the sport ball 10 by the animated member 60 at the locations along the exterior surface of the casing 12 corresponding to the left two touch sensors 21. Thus, as depicted in FIG. 4B, the magnitude of the touch data 26 generated by the left two touch sensors 21 is greater than the magnitude of the touch data 26 generated by the right two touch sensors 21.

As mentioned above, in various instances, after one or more touch sensors 21 disposed within a sport ball 10 generate touch data 26, the touch data 26 is made available to a processor 22. The processor 22 can then use the touch data 26 to determine a) if and when the sport ball 10 is impacted; b) how hard the sport ball 10 is impacted; and/or c) what the sport ball 10 is impacted by, e.g., a surface 30 or an animated member 60. For example, in some instances, the processor 22 can determine a time at which the touch data 26 was generated and, using the touch data 26 and the time at which the touch data 26 was generated, output an impact indication 27 indicating that the sport ball 10 was impacted and/or when the sport ball 10 was impacted. The processor 22 may also use the touch data 26 to determine how hard the sport ball 10 was impacted, such as by converting the touch data 26 into a force applied to the sport ball 10. For example, the touch data 26 may include a voltage or a change in resistance generated by an electrochemical touch sensor 21, as described above, and the processor 22 may possess or otherwise obtain information relating the voltage or the change in resistance to an equivalent amount of force applied to the electromechanical touch sensor 21. The amount of force with which a sport ball 10 is impacted may be referred to as an “impact force.”

Or for example, in some instances, after receiving, accessing, or otherwise obtaining touch data 26 generated by one or more touch sensors 21 disposed within a sport ball 10, a processor 22 can use the touch data 26 to generate an impact map 61, as depicted in FIGS. 3B and 4B. In this example, the processor 22 can then use characteristic features of the impact map 61 to make various determinations. For example, in some instances, the processor 22 can use the shape of the impact map 61 (e.g., the locations of touch sensors 21 with a lattice 29 of touch sensors 21 corresponding to locations along the exterior surface of the casing 12 of the sport ball 10 at which the sport ball 10 was impacted) to determine a shape or a partial shape of an object that impacted the sport ball 10. The processor 22 may also use the magnitude of the touch data 26 generated by each touch sensor 21 included in the impact map 61 in conjunction with the shape of the impact map 61 to determine the shape of an object that impacted the sport ball 10. For example, in the example depicted in FIG. 3B, the impact map 61, viewed from above or below, would appear as a set of concentric circles, with the magnitudes of the touch data 26 generated by the touch sensors 21 included in the impact map 61 evenly decreasing in any radial direction moving out from the center of the concentric circles. With this information, the processor 22 may be able to determine that the shape of the object that impacted the sport ball 10 is flat. Or for example, in the example depicted in FIG. 4B, the impact map 61, viewed from the side, would appear more like a rounded rectangle or an oval, with the magnitudes of the touch data 26 generated by the touch sensors 21 included in the impact map 61 decreasing more quickly along one axis than another. With this information, the processor 22 may be able to determine that the shape of the object that impacted the sport ball 10 is smaller and more pointed or rounded. Similarly, the processor 22 may use touch data 26 or an impact map 61 to determine if a sport ball 10 was impacted by a surface 30 or an animated member 60.

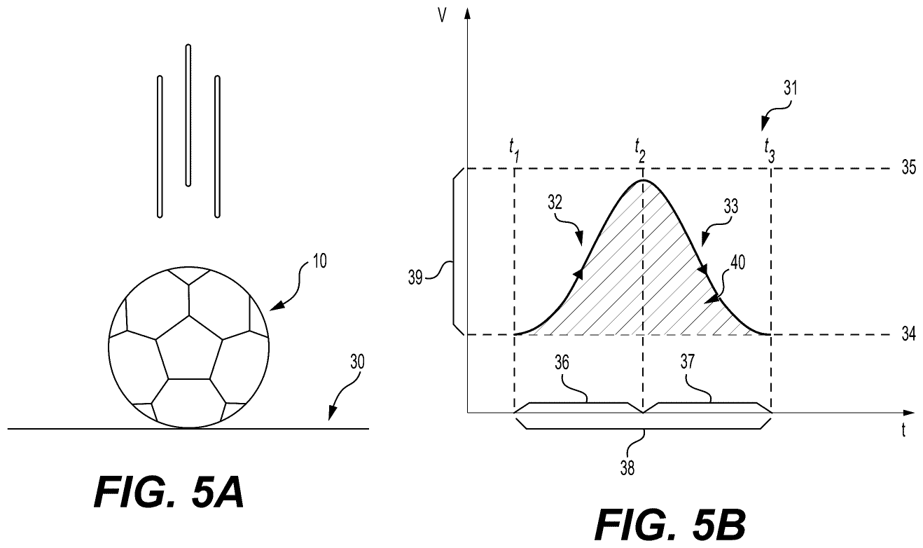

As mentioned above, in some instances, a sport ball 10 includes a touch sensing layer 28 is formed entirely or in part from a piezoelectric material, such that the touch sensing layer 28 itself may function as a single electromechanical touch sensor 21. In such an instance, when the sport ball 10 is impacted, no matter where the sport ball 10 is impacted, the piezoelectric material of the touch sensing layer 28 generates touch data in the form of a voltage in response to the impact. When the sport ball 10 is impacted, over a short period of time, the voltage generated by the touch sensing layer 28 in response to the impact may increase and decrease as the sport ball 10 deforms and returns to its resting shape. A graph of the voltage is plotted respect to time over this short period may be referred to as a touch hysteresis 31. FIG. 5B depicts a touch hysteresis 31 representing the changes over time in the voltage produced by a touch sensing layer 28 included in a sport ball 10 in response to an impact of the sport ball against a surface 30 (as illustrated in FIG. 5A). A touch hysteresis 31 may include a rising curve 32, representing the voltage increasing in response to the stimulus, and a falling curve 33, representing the voltage decreasing as the system returns to rest. A touch hysteresis 31 may include various characteristic features, such as a rest voltage 34, a peak voltage 35, a rising curve duration 36, a falling curve duration 37, a total duration 38, a voltage differential 39 (e.g., peak voltage 35 subtracted by rest voltage 34), an area 40 (e.g., an integral or a combined area beneath the rising curve 32 and the falling curve 33), a maximum slope of the rising curve 32 or the falling curve 33 (not shown), or any combination thereof. However, a touch hysteresis 31 may include any other characteristic feature. As depicted in FIG. 3B, a touch hysteresis 31 may have a form or shape similar to a bell curve, which may be substantially regular (e.g., the rising curve 32 and the falling curve 33 of the touch hysteresis 31 may be close to vertically symmetrical) or irregular (e.g., the rising curve 32 and the falling curve 33 of the touch hysteresis 31 may have completely different shapes).

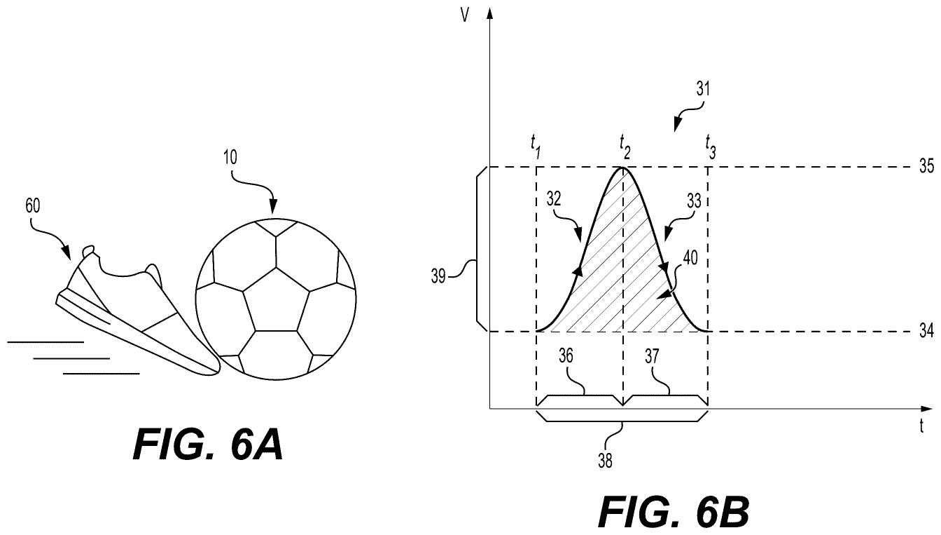

Similarly, FIG. 6B depicts a touch hysteresis 31 representing the changes over time in the voltage produced by a touch sensing layer 28 included in a sport ball 10 in response to an impact of the sport ball1 10 by an animated member 60 (as illustrated in FIG. 6A). As depicted in FIG. 6B, the total duration 38 of the touch hysteresis 31 is shorter than that of the touch hysteresis 31 in FIG. 5B, but the peak voltage 35 is greater than that of the touch hysteresis 31 in FIG. 5B. One or more relationships or ratios may be calculated using the characteristic features of a touch hysteresis 31, e.g., a ratio of the area 40 to the total duration 38 of the touch hysteresis 31, a ratio of the peak voltage 35 to the area 40 of the touch hysteresis 31, a ratio of the area under the rising curve 32 (not shown) to the area under the falling curve 33 (not shown) of the touch hysteresis 31, or a ratio of the maximum slope of the rising curve 32 to the total duration 38 of the touch hysteresis 31. However, any other suitable relationship or ratio may be calculated using the characteristic features of a touch hysteresis 31. In some instances, a touch sensing layer 28 included in a sport ball 10 may be formed entirely in part by a conductive material configured through which a standing current is passed, and a touch sensor 21 included in the sport ball 10 may be configured to measure a change in the resistance of the conductive material to a physical force. In such in instance, in much the same way that a touch hysteresis 31 may be created by plotting the changes over time in the voltage produced by a touch sensing layer 28 formed of a piezoelectric material, a touch hysteresis 31 may be created by plotting the changes over time in the resistance of the conductive material.

As mentioned above, in various instances, after a touch sensor 21 or a touch sensing layer 28 disposed within a sport ball 10 generates touch data 26, the touch data 26 is made available to a processor 22. The processor 22 can then use the touch data 26 to determine a) if and when the sport ball 10 is impacted; b) how hard the sport ball 10 is impacted; and/or c) what the sport ball 10 is impacted by, e.g., a surface 30 or an animated member 60. For example, in some instances, after receiving, accessing, or otherwise obtaining touch data 26 generated by a touch sensing layer 28 disposed within a sport ball 10, a processor 22 can use the touch data 26 to generate a touch hysteresis 31. In this example, the processor 22 can then use characteristic features of the touch hysteresis 31 to make various determinations. For example, in some instances, the processor 22 can use the touch hysteresis 31 to determine if and when the sport ball 10 is impacted by identifying the time (e.g., t₁) at which the voltage within the sport ball 10 increased significantly (e.g., by more than 1 or 2 percent), or at a significant rate (e.g., by more than 1 percent per millisecond). Or for example, in some instances, the processor 22 can use the touch hysteresis 31 to determine how hard the sport ball 10 is impacted by identifying the peak voltage 35 and using the peak voltage 35 to calculate a force with which the sport ball 10 was impacted. The force with which a sport ball 10 was impacted may be used to calculate or determine a speed (e.g., a ball speed) at which the sport ball 10 moved in response to the impact.

Or for example, the processor 22 can use the touch hysteresis 31 to determine whether the sport ball 10 was impacted by a surface 30 or by an animated member 60. As depicted in FIGS. 5B and 6B, the rising curve duration 36 of a touch hysteresis 31 representing an impact of a sport ball 10 against a surface 30 may be significantly longer in proportion to the total duration 38 than those of a touch hysteresis 31 representing an impact of the sport ball 10 by an animated member 60. Thus, in some instances, a processor 22 can use a touch hysteresis 31 to determine whether a sport ball 10 was impacted by a surface 30 or an animated member 60 by comparing the rising curve duration 36 to the total duration 38 of the touch hysteresis 31, such as by dividing the rising curve duration 36 by the total duration 38, and then comparing the result to a threshold percentage. For example, the threshold percentage may be 5%, 10%, 15%, etc. In such an instance, if the rising curve duration 36 divided by the total duration 38 is less than the threshold percentage, the processor 22 can determine that the sport ball 10 was impacted by an animated member 60. Or, in such an instance, if the rising curve duration 36 divided by the total duration 38 is greater than the threshold percentage, the processor 22 can determine that the sport ball 10 was impacted against a surface 30.

In another example, the processor 22 can use a touch hysteresis 31 to determine whether the sport ball 10 was impacted by a surface 30 or by an animated member 60 by analyzing the slope of the rising curve 32 and/or the slope of the falling curve 33. As depicted in FIGS. 5B and 6B, the slope of the rising curve 32 of a touch hysteresis 31 representing an impact of a sport ball 10 by an animated member 60 may be steeper than that of a touch hysteresis 31 representing an impact of the sport ball 10 by a surface 30. Thus, in some instances, for example, a processor 22 can calculate an initial derivative (e.g., at time t₁) of the rising curve 32 of a touch hysteresis 31 and compare the initial derivative to a slope threshold. In such an instance, if the initial derivative is greater than slope threshold, the processor 22 can determine that the sport ball 10 was impacted by an animated member 60. Or, in such an instance, if the initial derivative is less than the slope threshold, the processor 22 can determine that the sport ball 10 was impacted against a surface 30. However, a processor 22 may use any other aspects of touch data 26 or any other characteristic features of a touch hysteresis 31 to determine whether a sport ball 10 was impacted by a surface 30 or by an animated member 60.

In some instances, a processor 22 includes or is otherwise operative to access a correlation engine that can be used to determine whether a sport ball 10 was impacted by a surface 30 or by an animated member 60. For example, the correlation engine may include one or more machine learning algorithms that can receive touch data 26 generated by one or more touch sensors 21 and use the touch data 26 to determine whether a sport ball 10 was impacted by a surface 30 or by an animated member 60. In some instances, a processor 22, such as by employing a correlation engine, can determine a type of surface 30 or animated member 60 that a sport ball 10 was impacted by. For example, the processor 22 may be able to determine a shape, or a partial shape, of a surface 30 or an animated member 60 that impacted that the sport ball 10. Or for example, in some instances, if the processor 22 determines that the sport ball 10 was impacted against a surface 30, the processor 22 may also determine whether the surface 30 was the pitch of a soccer field or the goalpost of a soccer goal. Or for example, in some instances, if the processor 22 determines that the sport ball 10 was impacted by an animated member 60, the processor 22 may also determine whether the sport ball 10 was impacted by a hand, a foot, or a head. In some instances, a processor 22 determines a confidence or a likelihood of a sport ball 10 having been impacted by a surface 30 or by an animated member 60. For example, in some instances, a processor 22 determines that there is an X% likelihood that a sport ball 10 has been impacted by an animated member 60. In some instances, a processor 22 determines that there is an X% likelihood that a sport ball 10 has been impacted by an animated member 60, and, accordingly, that there is a (100-X)% likelihood that the sport ball 10 has been impacted by a surface 30.

The impact of a sport ball 10 against another object, represented by the touch data 26 generated in response to the impact (e.g., the touch data 26 included in an impact map 61 or a touch hysteresis 31), may be referred to as a “touch event.” In some instances, when analyzing the touch data 26 of a touch event (e.g., to determine if, when, or by what a sport ball 10 was impacted), the processor 22 can factor in or otherwise incorporate touch data 26 representing one or more previous touch events. For example, in some instances, when analyzing the touch data 26 of a second touch event that occurred shortly after a first touch event, if the processor 22 determined that the first touch event was an impact of the sport ball 10 by an animated member 60, the processor 22 may increase the likelihood that the second touch event was an impact of the sport ball 10 against a surface 30, or vice versa. However, the processor 22 may use the touch data 26 of a prior touch event, or any information that can be gleaned from a corresponding impact map 61 (e.g., the shape of the impact map) or touch hysteresis 31, in any other way when analyzing the touch data 26 of a subsequent touch event.

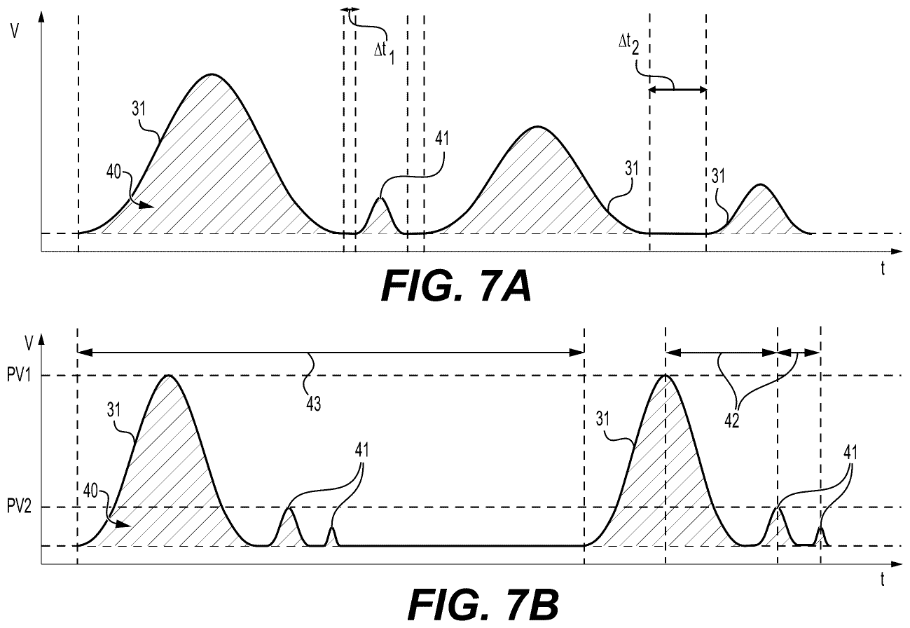

A fluctuation in the voltage or resistance within a sport ball 10 may not always be a touch hysteresis 31 representing an impact of the sport ball 10. For example, a fluctuation in the voltage or resistance within the sport ball 10 may be a reverberation 41. A reverberation 41 may be a fluctuation in the voltage or resistance within a sport ball 10 in response to an initial increase in the voltage or resistance due to the sport ball 10 being impacted (e.g., by a surface 30 or an animated member 60). In some instances, a touch event includes only a touch hysteresis 31 representing an impact of the sport ball 10. In some instances, a touch event includes a touch hysteresis 31 representing an impact of the sport ball 10 and one or more reverberations 41 that follow the touch hysteresis 31 in response to the impact of the sport ball 10. FIGS. 7A and 7B depict touch hystereses 31 followed by reverberations 41. In the example depicted by FIG. 7A, each of the three touch hystereses 31 represent a separate impact of a sport ball 10 by a surface 30 (e.g., the sport ball 10 bounced on the surface 30 three times). The first touch hysteresis 31 (i.e., the leftmost touch hysteresis 31) is followed by a reverberation 41. The second and third touch hystereses 31 are not followed by a reverberation 41. This may be because, for example, in a first bounce off the surface 30 represented by the first touch hysteresis 31, the sport ball 10 bounced high enough and/or remained suspended in the air long enough for a reverberation 41 to be registered by a touch sensing layer 28 disposed within the sport ball 10, but in subsequent second and third bounces, represented by the second and third touch hystereses 31, respectively, the sport ball 10 does not bounce high enough or remain suspended in the air long enough for a reverberation to be registered by the touch sensing layer 28.

In some instances, to determine whether a fluctuation in voltage or resistance within a sport ball 10 is a touch hysteresis 31 representing an impact of the sport ball 10 (e.g., by a surface 30 or an animated member 60) or a reverberation 41, a processor 22 can use or otherwise consider touch data 26 from a time horizon extending beyond the fluctuation in voltage or resistance. For example, to determine that the fluctuation represented by reverberation 41 in FIG. 7A is a reverberation 41 and not a touch hysteresis 31 representing an impact of the sport ball 10, a processor 22 can compare an area under the curve of the fluctuation to an area 40 under the curve of a touch hysteresis 31 that precedes the fluctuation, such as by dividing the area 40 under the curve of the touch hysteresis 31 by the area under the curve of the fluctuation. If the result is greater than or equal to a threshold value, the processor 22 can identify the fluctuation as a reverberation 41 and not a touch hysteresis 31 that represents an impact of sport ball 10. Conversely, if the result is less than the threshold value, the processor 22 can identify the fluctuation as a touch hysteresis 31 representing an impact of the sport ball 10 (e.g., by a surface 30 or an animated member 60). Or for example, to determine that the fluctuation represented by reverberation 41 in FIG. 7A is a reverberation 41 and not a touch hysteresis 31 representing an impact of the sport ball 10, a processor 22 can determine or calculate an amount of time between the fluctuation and a touch hysteresis 31 that precedes the fluctuation, e.g., Δt₁. If the amount of time between the fluctuation and the touch hysteresis 31 that precedes the fluctuation is less than or equal to a threshold amount of time, the processor 22 can identify the fluctuation as a reverberation 41 and not a touch hysteresis 31 representing an impact of the sport ball 10. Conversely, if the amount of time between the fluctuation and the touch hysteresis 31 that precedes the fluctuation is greater than the threshold amount of time, the processor 22 can identify the fluctuation as a touch hysteresis 31 representing an impact of the sport ball 10 (e.g., by a surface 30 or an animated member 60). For example, Δt₁ may be less than the threshold amount of time, and Δt₂ may be greater than the threshold amount of time; therefore, a processor 22 may identify the fluctuation represented by reverberation 41 as a reverberation 41 and the fluctuation represented by the third touch hysteresis 31 as a touch hysteresis 31 representing an impact of the sport ball 10. However, a processor 22 may use touch data 26 from a time horizon expanding beyond a fluctuation in voltage or resistance within a sport ball 10 to determine if the fluctuation is a reverberation 41 or a touch hysteresis 31 representing an impact of the sport ball 10 in any other way.

In the example depicted in FIG. 7B, a first touch hysteresis 31 representing a first impact of the sport ball 10 with an animated member 60 is followed by two reverberations 41, and a second touch hysteresis 31 representing a second impact of the sport ball 10 with an animated member 60 is also followed by two reverberations 41 (e.g., the sport ball 10 is kicked into the air, where it reverberates, and then, before the sport ball 10 is allowed to land on a surface 30, the sport ball 10 is kicked back into the air, where it reverberates again). In this example, a processor 22 may determine that the fluctuation represented by the first reverberation 41 following the first touch hysteresis 31 is a reverberation 41 and not a touch hysteresis 31 representing an impact of the sport ball 10 by comparing the peak voltage of the fluctuation (e.g., PV2) with the peak voltage of the touch hysteresis 31 that precedes the fluctuation (e.g., PV1), such as by dividing the peak voltage of the preceding touch hysteresis 31 by the peak voltage of the fluctuation. If the result is greater than or equal to a threshold value, the processor 22 can determine that the fluctuation is a reverberation 41. Conversely, if the result is less than the threshold value, the processor 22 can determine that the fluctuation is touch hysteresis 31 representing an impact of the sport ball 10. Or for example, a processor 22 may determine that the fluctuation represented by the first reverberation 41 following the second touch hysteresis 31 is a reverberation 41 and not a touch hysteresis 31 representing an impact of the sport ball 10 by determining and/or comparing one or more oscillation frequencies 42 between the fluctuation and the touch hysteresis 31 that precedes the fluctuation and/or between the fluctuation and the fluctuation that follows the fluctuation (e.g., the fluctuation represented by the second reverberation 41 following the second touch hysteresis 31). An oscillation frequency 42 may be the amount time between the peak voltages of two consecutive fluctuations. In this example, because the oscillation frequencies 42 between the fluctuation and the fluctuations immediately preceding and immediately following the fluctuation are less than a threshold oscillation frequency, the processor 22 can determine that the fluctuation is a reverberation 41, and not a touch hysteresis 31 representing an impact of the sport ball 10. After identifying two consecutive touch hystereses 31 representing two consecutive impacts of a sport ball 10 (e.g., the two touch hystereses 31 depicted in FIG. 7B), a processor 22 can identify an amount of time between two impacts as a time of flight 43. In some instances, a processor 22 only identifies an amount of time between two impacts as a time of flight 43 if there are a threshold number of reverberations 41 between the two impacts.

In some instances, when analyzing the touch data 26 of a touch event, the processor 22 can factor in or otherwise incorporate externally sourced information. For example, in some instances, a correlation engine included in or otherwise accessible by the processor 22 includes historical touch data 26 generated by a plurality of touch sensors 21 disposed within a respective plurality of sport balls 10 during a multitude of prior touch events. In such an instance, the processor 22 can use the historical touch data 26 when analyzing the touch data 26 of a recent touch event. Or for example, in some instances, the processor 22 can receive or otherwise access user submitted information and use the user submitted information when analyzing the touch data 26 of a touch event. For example, in some instances, after the processor 22 determines that a sport ball 10 was impacted by a surface 30 or an animated member 60, a user of the touch sensing sport ball system 20 can confirm or deny the processor's determination, such as through the use of a graphical user interface (GUI) 25 provided by the touch sensing sport ball system 20, as described above and below, thereby providing the processor 22 with feedback that the processor 22 can use when analyzing the touch data 26 of a subsequent touch event, such as by training a machine learning algorithm included in a correlation engine included in or otherwise accessible by the processor 22. Or for example, in some instances, a user of the touch sensing sport ball system 20 can submit to the processor 22, such as through the use of a GUI 25 provided by the touch sensing sport ball system 20, a type of surface that the sport ball 10 will be utilized on (e.g., grass, turf, or asphalt). In such an instance, the processor 22 can use knowledge of the type of surface that the sport ball 10 will be utilized on when analyzing the touch data 26 of a touch event. However, the processor 22 may use any externally sourced information in any other way when analyzing the touch data 26 of a touch event.

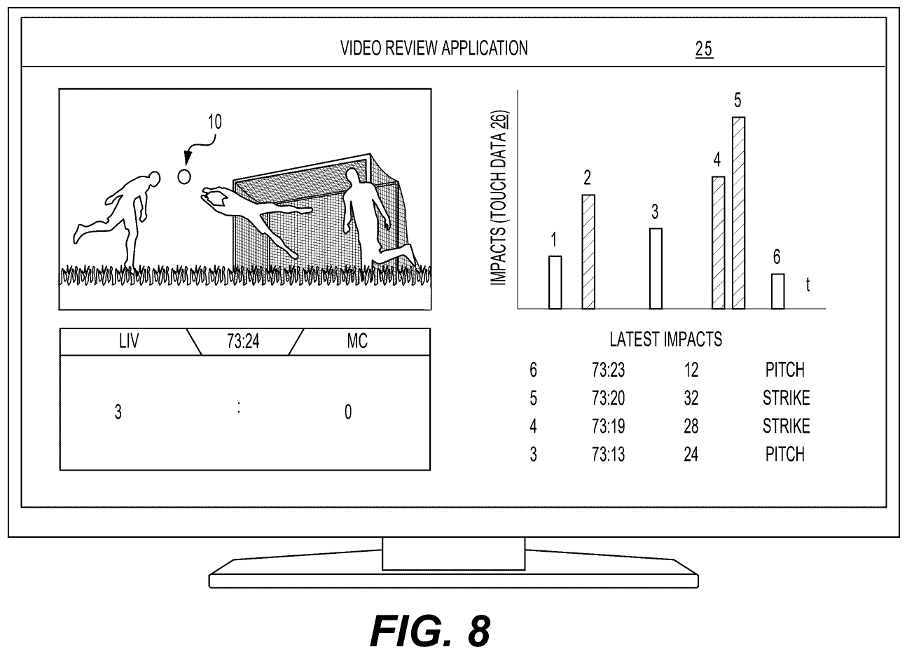

As mentioned above, in various instances, a processor 22 is operative to receive, access, or otherwise obtain touch data 26 generated by one or more touch sensors 21 disposed within a sport ball 10 and, using or based on the touch data 26, determine a) if and when the sport ball 10 is impacted; b) how hard the sport ball 10 is impacted; and/or c) what the sport ball 10 is impacted by, e.g., a surface 30 or an animated member 60. As mentioned above, in some embodiments, the processor 22 is further operative to cause a graphical user interface (GUI) 25 to display the touch data 26 or an impact indication 27. FIG. 8 illustrates a GUI 25 accessed or provided by a touch sensing sport ball system 20. In the example illustrated in FIG. 8, the GUI 25 is a video review application used by soccer referees, e.g., a video assisted referee application. In this example, the sport ball 10, a soccer ball, includes one or more touch sensors 21 and a communication component 24 disposed within the sport ball 10, e.g., disposed within a pocket 17 included in a bladder 14 of the sport ball 10. In this example, the one or more touch sensors 21 are electromechanical touch sensors that generate touch data 26 by generating voltages or changes in resistance in response to the sport ball 10 being impacted, and the communication component 24, communicatively coupled to the one or more touch sensors 21, wirelessly transmits the touch data 26 generated by the touch sensor 21 to a remote processor 22 instantly and in real-time. In this example, the processor 22 uses the touch data 26 to determine a) if and when the sport ball 10 is impacted; b) how hard the sport ball 10 is impacted; and c) what the sport ball 10 is impacted by, e.g., a soccer field (or “pitch”) or a soccer player (or “strike”). For example, as illustrated in FIG. 8, in the ten seconds between 73:13 and 73:23 of a soccer game in which the sport ball 10 is being utilized, the processor 22 has determined that the sport ball 10 was impacted four times (i.e., impacts 3, 4, 5, and 6). The processor 22 has also caused the GUI 25 to display the latest six impacts involving the sport ball 10, along with an impact force for each impact, and whether the impact was determined to have been with the pitch or by a strike. In this example, the processor 22 has caused the GUI 25 to display the latest impacts involving the sport ball 10 in real-time. The GUI 25 can then be used by a referee of the soccer game to help the referee determine whether the soccer ball was struck by a player while the player was offside, or if the soccer ball was struck by a hand, for example.

A GUI 25, and the information generated for display within a GUI 25 by a processor 22, may take on many different forms based on a particular application. For example, when the sport ball 10 is a soccer ball designed or otherwise intended for individual youth soccer practice, the GUI 25 may be a simple interface that displays information such as ball speed (e.g., calculated by a processor 22 using peak voltage values), time of flight 43, and a number of consecutive impacts of the sport ball 10 by an animated member 60. Or for example, when the sport ball 10 is a volleyball designed or otherwise intended for use during competition, the GUI 25 may be a more intricate interface that displays information indicating whether an impact of the sport ball 10 was by a surface 30 or an animated member 60, information indicating whether an impact of the sport ball 10 was a serve, a set, a spike, or a dig. Or for example, when the sport ball 10 is a basketball, the GUI 25 may display information indicating whether an impact of the sport ball 10 was by a floor, a backboard, or a rim of a basket. However, the GUI 25 may take on any suitable form and display any suitable information for any suitable application.

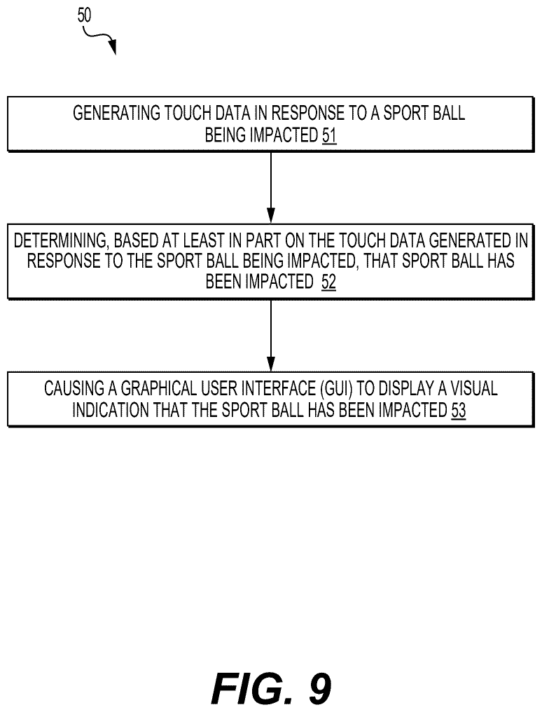

FIG. 9 depicts a flow diagram of a method 50 for determining that a sport ball 10 has been impacted. The method 50 may be performed by a touch sensing sport ball system 20, as described above. As depicted in FIG. 9, in some instances, the method 50 begins with steps 51 and 52, wherein the touch sensing sport ball system 20 generates touch data 26 in response to a sport ball 10 being impacted. For example, as described above, the touch sensing sport ball system 20 may include one or more electromechanical touch sensors 21 disposed within the sport ball 10 that generate voltages or changes in resistance in response to the sport ball 10 being impacted. In some instances, the touch data 26 is provided to a computer-readable memory 23 for storage. As depicted in FIG. 9, in some instances, after the touch sensing sport ball system 20 generates touch data 26, the method 50 continues with step 53, wherein the touch sensing sport ball system 20 determines, based at least in part on the touch data 26, that the sport ball 10 has been impacted. As depicted in FIG. 9, in some instances, after determining that the sport ball 10 was impacted, the method 50 continues with step 54, wherein the touch sensing sport ball system 20 causes a graphical user interface (GUI) 25 to display a visual indication indicating that the sport ball 10 was impacted. For example, as described above, a processor 22 can determine a) that the sport ball 10 was impacted; b) when the sport ball 10 was impacted; and c) how hard the sport ball 10 was impacted, and cause a GUI 25 accessed or provided by the touch sensing sport ball system 20 to display one or more visual indications of the sport ball 10 being impacted and how hard the sport ball 10 was impacted.

It will be apparent to those skilled in the art that various modifications and variations can be made to the disclosed system without departing from the scope of the disclosure. Other embodiments of the system will be apparent to those skilled in the art from consideration of the specification and practice of the system disclosed herein. It is intended that the specification and examples be considered as exemplary only, with a true scope of the disclosure being indicated by the following claims and their equivalents.

We claim:

1. A touch sensing sport ball system, the system comprising:
a casing that forms an exterior layer of a sport ball;
a bladder disposed within the casing;
a touch sensing layer disposed between the exterior layer and the bladder and operative to generate touch data when the sport ball is impacted; and
at least one processor operative to:
access the touch data generated by the touch sensing layer;
determine, based at least in part on the touch data, that the sport ball has been impacted; and
output an indication that the sport ball has been impacted.

2. The touch sensing sport ball system of claim 1, wherein the at least one processor is further operative to:
determine, based at least in part on the touch data, an amount of force with which the sport ball was impacted; and
output an indication of the amount of force with which the sport ball was impacted.

3. The touch sensing sport ball system of claim 2:
wherein the touch sensing layer comprises a piezoelectric material and is further operative to generate the touch data by generating a voltage in response to the sport ball being impacted; and
wherein the at least one processor is further operative to determine the amount of force with which the sport ball was impacted based at least in part on the voltage generated in response to the sport ball being impacted.

4. The touch sensing sport ball system of claim 1, wherein the at least one processor is further operative to:
determine, based at least in part on the touch data, a shape of an object that impacted the sport ball; and
output an indication of the shape of the object that impacted the sport ball.

5. The touch sensing sport ball system of claim 4:
wherein the touch sensing layer comprises a piezoelectric material and is further operative to generate the touch data by generating, in response to the sport ball being impacted, a plurality of voltages corresponding to a respective plurality of locations along the casing at which the sport ball was impacted; and
wherein the at least one processor is further operative to determine the shape of the object that impacted the sport ball based at least in part on the plurality of voltages generated in response to the sport ball being impacted.

6. The touch sensing sport ball system of claim 4, wherein the at least one processor is further operative to:
determine, based at least in part on the shape of the object that impacted the sport ball, whether the sport ball was impacted by a surface or an animated member; and
output an indication of the determination of whether the sport ball was impacted by a surface of an animated member.

7. The touch sensing sport ball system of claim 1:
further comprising a graphical user interface (GUI) executed on a computing device; and
wherein the at least one processor is further operative to cause the GUI to display the indication that the sport ball has been impacted.

8. The touch sensing sport ball system of claim 7, wherein the at least one processor is further operative to cause the GUI to display an indication of a shape of an object that impacted the sport ball.

9. The touch sensing sport ball system of claim 7, wherein the at least one processor is further operative to cause the GUI to display an indication of a determination of whether the sport ball was impacted by a surface or an animated member.

10. The touch sensing sport ball system of claim 1, wherein the at least one processor is housed within a pocket disposed within the bladder.

11. A method for analyzing an impact of a sport ball, the method comprising:
generating touch data in response to a sport ball being impacted;
determining, based at least in part on the touch data generated in response to the sport ball being impacted, that sport ball has been impacted; and
causing a graphical user interface (GUI) to display a visual indication that the sport ball has been impacted.

12. The method of claim 11, further comprising:
determining, based at least in part on the touch data generated in response to the sport ball being impacted, an amount of force with which the sport ball was impacted; and
causing the GUI to display a visual indication of the amount of force with which the sport ball was impacted.

13. The method of claim 12, further comprising:
generating the touch data by generating a voltage in response to the sport ball being impacted; and
determining, based at least in part on the voltage generated in response to the sport ball being impacted, the amount of force with which the sport ball was impacted.

14. The method of claim 11, further comprising:
determining, based at least in part on the touch data generated in response to the sport ball being impacted, a shape of an object that impacted the sport ball; and
causing the GUI to display a visual indication of the shape of the object that impacted the sport ball.

15. The method of claim 14, further comprising:
generating the touch data by generating, in response to the sport ball being impacted, a plurality of voltages corresponding to a respective plurality of locations along a casing of the sport ball at which the sport ball was impacted; and
determining, based at least in part on the plurality of voltages generated in response to the sport ball being impacted, the shape of the object that impacted the sport ball.

16. A touch sensing sport ball apparatus, comprising:
a casing that forms an exterior layer of a sport ball;
a bladder disposed within the casing; and
a touch sensing layer disposed between the exterior layer and the bladder and operative to generate touch data when the sport ball is impacted that can be used by at least one processor to determine that the sport ball has been impacted.

17. The touch sensing sport ball apparatus of claim 16, further comprising the at least one processor, wherein the at least one processor is housed within a pocket disposed within the bladder.

18. The touch sensing sport ball apparatus of claim 16, further comprising a wireless communication component operative to transmit the touch data generated by the touch sensing layer to the at least one processor.

19. The touch sensing sport ball apparatus of claim 16, wherein the touch sensing layer comprises a piezoelectric material operative to generate the touch data by generating a voltage in response to the sport ball being impacted.

20. The touch sensing sport ball apparatus of claim 19, wherein the touch sensing layer entirely covers an interior surface of the casing.
